(19) United States
(12) Patent Application Publication
(10) Pub. No.: US 2021/0401730 A1
(43) Pub. Date: Dec. 30, 2021

(54) USE OF EXTRACTS OF AGAVE FOR A HAIR APPLICATION

(71) Applicant: LABORATOIRES DE BIOLOGIE VEGETALE YVES ROCHER, LA GACILLY (FR)

(72) Inventors: Céline LAPERDRIX, SAINT-REMY-LÈS-CHEVREUSE (FR); Christian LUBRANO, SÈVRES (FR); Sophie ZOZINE, FONTENAY-LE-FLEURY (FR)

(21) Appl. No.: 16/492,599

(22) PCT Filed: Mar. 6, 2018
(86) PCT No.: PCT/FR2018/050506

§ 371 Date: Aug. 24, 2021

(30) Foreign Application Priority Data
Mar. 7, 2017 (FR) 1751832

(51) Int. Cl. A61K 8/9794; A61K 8/73; A61Q 7/00

(52) U.S. Cl. CPC A61K 8/9794; A61K 2800/805; A61Q 7/00; A61K 8/73

(57) ABSTRACT

The invention relates to the non-therapeutic cosmetic use of an extract of agave for improving hair growth and/or the appearance of the hair and/or of the scalp. The invention also relates to a cosmetic or dermatological composition which improves hair growth and/or the appearance of the hair and/or of the scalp, comprising extracts of Agave tequilana and a cosmetically and/or dermatologically acceptable carrier, said composition comprising from 0.05% to 5% by weight of said extracts of Agave tequilana relative to the total weight of the composition, and said extract of agave comprising fructooligosaccharides which have a degree of polymerization of less than or equal to 10 and/or inulins which have a degree of polymerization of from 10 to 60. The invention also relates to a device which is in a form chosen from a jar, a bottle, a pump-dispenser bottle, a mask and a spray, said device comprising a composition of the invention, and also to a non-therapeutic cosmetic method for improving hair growth and/or the appearance of the hair and/or of the scalp, comprising an application of the cosmetic composition of the invention to the hair and/or the scalp.

### TECHNICAL FIELD

The present invention relates to a cosmetic or dermatological composition comprising agave extracts, and also to a non-therapeutic cosmetic use of an agave extract for improving the growth of the hair and/or the appearance of the hair and/or of the scalp.

The present invention has an application in the field of cosmetics and dermatology, more particularly of hair cosmetics.

In the description below, the references between square brackets ([ ]) refer to the list of references presented at the end of the text.

### PRIOR ART

The skin is a vital organ in its own right which is composed of three distinct tissues, each taking on different roles by virtue of different cell types and different structures.

The tissue most at the surface, and therefore the most exposed, is the epidermis. This pluristratified (Malpighian) and keratinized epithelium, the outermost portion of which is the stratum corneum, is composed of different cells associated with numerous barrier and protective functions. The predominant cells are keratinocytes which, through their processes of proliferation/differentiation, lead to the formation of the stratum corneum, known for its hydrophobic properties and its compact and leaktight appearance. The major role of the epidermis is to provide the skin, and therefore the human body, with a first line of protection against external attacks, such as physical, chemical, hydric, and bacteriological attacks, especially via differentiation and the stratum corneum.

In the intermediate position, the dermis is also a connective tissue, composed predominantly of fibroblasts and matrix proteins, giving the skin its known qualities of compressibility and elasticity. Within the connective web, other cells and structures are also inserted, such as a significant circulatory and nutritive network, consisting of blood vessels and lymphatic capillaries, and also the epidermal annexes: head hair, body hair, nails, pilosebacious glands and sweat glands, which originate in the deep dermis.

The hypodermis, located at depth and predominantly consisting of fat lobules (adipocytes), provides a function of primary support, of mechanical and heat protection, and also serves for storage of energy reserves which can be rapidly mobilized for all biological requirements, for instance cell renewal, defense of the organism or muscle contraction.

The scalp is the part of the skin located on the head which grows a specific type of hair: head hair. Among the skin annexes, hair follicles have the particular feature of being a tubular invagination of the epidermis which extends into the dermis or even the hypodermis. This epidermal invagination expands at its inner end to form a bulb composed of epithelial cells, keratinocytes which will proliferate and keratinize to form the hair. The life cycle of hair comprises three phases: the growth phase, referred to as the anagen phase (lasts 3 to 5 years), the phase of degradation until expulsion, referred to as the catagen phase (lasts 15 to 20 days), then the resting phase, referred to as the telogen phase (lasts 2 to 3 months). The growth of the hair is possible by virtue of interactions between the keratinocytes, the fibroblasts of the dermal papilla and the endothelial cells of the vessels present in this region. Indeed, during the anagen phase, signals are sent by the fibroblasts of the dermal papilla to stimulate the process of angiogenesis which will make it possible to feed the birth, then the growth, of the hair. The migration of endothelial cells towards the bulb of the hair follicle and the formation of vessels therefore have important roles in this growth phase.

Due to their outward position and their long lifetime, hairs accumulate damage caused by environmental stress such as UV radiation, pollution, cold, but also esthetic treatments (drying, curling, straightening, dyeing, etc.), or the use of aggressive products (too frequent or unsuitable shampooing operations). Moreover, they are affected by internal factors such as stress, hormonal modifications or else the type of food.

There are currently few cosmetic products that stimulate the growth of the hair, and these are mainly hormonal treatments, dietary supplements or derivatives of amino acids (taurine or arginine) resulting from non-natural biotechnology processes.

There is therefore a real need to find novel compositions and/or compounds of plant origin and/or which are natural, making it possible to effectively ensure and/or improve the growth, shine and softness of the hair.

### DESCRIPTION OF THE INVENTION

The aim of the present invention is specifically to meet these requirements and respond to these drawbacks of the prior art.

The inventors are the very first to use agave extracts, specifically making it possible to effectively meet the abovementioned requirements.

Agave is a monocotyledonous plant currently classified in the family of the Agavaceae. The most common species are Agave tequilana, Agave americana and Agave attenuata. Agave is endemic on the American continent and is present from the south of Canada to the north of South America and the Caribbean Islands, in particular in Mexico and throughout Central America (Maria de la Soledad Alonso Gutierrez, thesis: “Valorisation de la bagasse de l'Agave tequilana W. cv azul: caractérisation, étude de la digestibilité et de la fermentation des sucres” [Utilizing bagasse of Agave tequilana W. cv azul: characterization, study of digestibility and of sugar fermentation], 2005 MD).

Agaves have an underground part having the form of a rhizome, and an aerial part having long stems and large fibrous leaves, arranged in the form of a rosette and tipped with a thorn. The flowers are located at the end of the stem and are greeny yellow in color ([1]). Inulin is a storage polysaccharide found in plants of the family Asteraceae, especially in the bulbs and roots of dahlias, Jerusalem artichokes, artichokes, dandelions and chicory, the latter being the source most widely used industrially. Inulin consists of 30 to 40 fructose units connected by 13 (1-2) glycosidic linkages and of one or two glucose molecules placed at the chain end, and is in reality a glucofructosan. Agave inulins are carbohydrate reserves of fructan type (also referred to as fructosans). Polyfructosans were extracted from 8 year old Agave tequilana Weber var. azul. The result of the characterization of these fructosans gave a degree of polymerization of between 3 and 29, and a complex composition of fructooligosaccharides with β (1-2) and β (2-6) linkages, different from the inulins encountered in other plants (López, M. G.; Mancilla, N. A.; Mendoza-Diaz, G. (2003).: Molecular structures of fructans from Agave tequilana Weber var. azul. J. Agric. Chem. 51, 7835-7840 ([2]); MD). Chicory inulins have a different structure from that of agave inulins. Indeed, chicory inulins have linear chains of fructoses connected by beta 2-1 linkages whereas agave inulins, even though they are also mainly composed of fructose, have lateral structures branched from the main structure by beta 2-6 linkages (López, M. G. et al. ([2])).

The inventors are the very first to have discovered that agave extracts unexpectedly have an effect on improving the growth and the appearance of the hair and of the scalp.

Indeed, the inventors have demonstrated an effect of extracts of agave, especially of Agave tequilana, on the formation of blood vessels and the migration of endothelial cells, for an effect of stimulation of the growth of the hair follicle.

Moreover, the inventors have demonstrated an effect of agave extracts on the stimulation of the renewal of epidermal keratinocytes, for an effect on the shine and the appearance of the hair and of the scalp.

Thus, a first subject of the invention relates to the non-therapeutic cosmetic use of an agave extract for improving the growth of the hair and/or the appearance of the hair and/or of the scalp.

“Improving the growth of the hair” is intended to mean any improvement, whether qualitative and/or quantitative, in the vitality and/or growth of the hair, in terms of length and/or in terms of diameter. These improvements can be measured by any method for measuring the growth or the density of the hair, or the phase distribution thereof, which are known to those skilled in the art, for example by means of a trichogram comprising taking a sample, using a sharp tug, of approximately 40 hairs over 3 specific zones of the scalp in order to study the roots and the diameter of the hair under the microscope or on a microfiche reader, or by means of macro photography with shaving of a small region which is subsequently photographed using a video camera, to count the exact number of hairs and monitoring with specialized software, or by means of a phototrichogram comprising the use of specialized software (Tricoscan™) to count the total number of hairs, after carrying out macro photography with shaving.

“Improving the appearance of the hair” is intended to mean any qualitative and/or quantitative improvement in the optical reflective properties and/or tactile softness properties and/or physical regularity properties of the hair in general. These improvements may be measured by any measurement method known to those skilled in the art, for example by reflectance or acoustic tribology measurements.

“Improving the appearance of the scalp” is intended to mean a qualitative and/or quantitative stimulation for renewing the epidermis of the scalp and/or an improvement in the homogeneity and regularity of the scalp. These improvements may be measured by any measurement method known to those skilled in the art, for example by standardized photographs produced on a stereotactic apparatus enabling, by virtue of a fixed distance and of a standardized illumination, overall photos of the scalp which are reproducible before or after treatment.

The cosmetic use of the invention may make it possible to give the hair shine and/or soften the hair and/or improve the radiance of the hair.

Advantageously, the agave extract may stimulate keratinocyte renewal and/or the formation of microtubules and/or the migration of endothelial cells. Without wishing to be bound by the explanation of a mechanism of action, it appears that the stimulation of keratinocyte renewal and/or the formation of microtubules and/or the migration of endothelial cells is the cause of the abovementioned effects.

For the purposes of the present invention, “agave extract” is intended to mean any agave extract making it possible to obtain at least one of the abovementioned effects. It may be an extract of Agave tequilana, of Agave americana or of Agave attenuata. Preferentially, it is an extract of Agave tequilana.

The extract of agave, especially of Agave tequilana, may comprise fructans. The fructans may comprise fructooligosaccharides having a degree of polymerization less than or equal to and/or inulins having a degree of polymerization greater than 10.

These fructans are thus derived directly from the plant, and are therefore natural, that is to say not chemically modified, especially not comprising any chemically grafted groups of hydrophobic nature.

The fructooligosaccharides may for example have a degree of polymerization from 3 to 10.

The inulins may have a degree of polymerization strictly greater than 10, for example of 10 to 60, or for example from 10 to 29.

The agave extract may comprise or consist solely of fructooligosaccharides having a degree of polymerization less than or equal to 10. Alternatively, the agave extract may comprise or consist solely of inulins having a degree of polymerization greater than 10. For example, the ratio of fructooligosaccharides to inulins may be from 100/0 to 0/100, the limit values 0/100 and 100/0 optionally not being included. The ratio may for example be 75/25, or 25/75, or 50/50.

Advantageously, an extract of inulins having a degree of polymerization greater than 10 may also have an effect of protection of the hair fiber by improving the hydrophobicity of the hair fiber. Indeed, the Applicant identified that such inulins have a hydrophobic action, enabling protection of the hair fiber, despite their hydrophilic nature. The use of these inulins thus makes it possible to improve the hydrophobic nature of the hair, especially by improving the hydrophobicity of the hair fiber.

The use of an agave extract comprising a ratio of fructooligosaccharides to inulins of 50/50 would thus make it possible to combine the effect of improving the growth of the hair and/or the appearance of the hair and/or of the scalp and that of protecting the hair fiber by improving the hydrophobicity of the hair fiber.

The agave extract may be obtained by extraction of the carbohydrates from the bagasse, clarification of the crude liquor obtained at the end of the extraction, then separation in order to obtain a fraction of fructans, especially of fructooligosaccharides, having a degree of polymerization less than or equal to 10 and/or a fraction of inulins having a degree of polymerization greater than 10.

An extract of Agave tequilana may be a commercial extract, for example the product Metlos® (NEKUTLI), characterized by fructooligosaccharides having a degree of polymerization less than or equal to 10, or the product Metlin® (NEKUTLI) characterized by true inulins having a degree of polymerization greater than 10. It may be a mixture of the products Metlos® and Metlin®, the ratio of which may be equal for example to 75/25, or to 25/75, or to 50/50.

Another subject of the invention relates to a cosmetic or dermatological composition, especially improving the growth and/or appearance of the surface of the hair and/or of the scalp, comprising extracts of Agave tequilana and a cosmetically and/or dermatologically acceptable carrier, said composition comprising from 0.05 to 5% by weight of agave extracts relative to the total weight of the composition, said agave extract comprising fructooligosaccharides having a degree of polymerization less than or equal to 10 and/or inulins having a degree of polymerization from 10 to 60. All the characteristics of agave extracts mentioned above for the use of the invention, and also all the characteristics of the use of an extract described above, apply mutatis mutandis to the composition of the invention.

In the present invention, “cosmetic composition” is intended to mean any composition intended for cosmetic, that is to say esthetic, use, a composition that may be brought into contact with the superficial parts of the human body, for example the epidermis and the body hair and head hair systems. Advantageously, a cosmetic composition exclusively or primarily makes it possible to protect them, fragrance them, keep them in a good state, modify their appearance or correct their superficial esthetic defects.

In the present document, “dermatological composition” is intended to mean any composition intended for dermatological use, that is to say a composition that may be brought into contact with the superficial parts of the human body for a treatment of the skin, especially of the scalp, and the integuments, such as head hair or body hair.

“Cosmetically or dermatologically acceptable carrier” is intended to mean a carrier suitable for use in contact with human and animal skin cells, in particular the cells of the epidermis, without toxicity, irritation, induced allergic reaction and the like, and adjusted to a reasonable advantage/risk ratio.

The cosmetically acceptable carrier may for example be chosen from water, glycerol, aloe vera gel, an aqueous plant extract, surfactants of natural origin, this list being nonlimiting.

Another subject of the invention relates to a process for preparing a cosmetic or dermatological composition as defined above, comprising the mixing of an agave extract as defined previously and of a cosmetically and/or dermatologically acceptable carrier.

The composition of the invention may be obtained by any suitable process known to those skilled in the art for the production of a cosmetic composition. This may for example be simple mixing. It may alternatively for example be a process comprising a step of incorporating an internal phase into an external phase by means of an emulsifier, for example a turbomixer of rotor-stator type. It may also be, for example, a process using the Phase Inversion Temperature (PIT) process, this process conventionally being used by those skilled in the art to obtain oil-in-water emulsions in which the dispersed droplets are particularly fine, for example with a diameter of from 0.1 to 1 μm.

According to the invention, the cosmetic or dermatological composition may comprise from 0.05 to 5% by weight of agave extracts relative to the total weight of the composition, for example from 0.1 to 5%, from 0.1 to 1%, from 0.25 to 1% or from 0.25 to 0.5%.

The cosmetic or dermatological composition of the present invention may be in any form suitable for a cosmetic or dermatological application. Advantageously, the composition is a composition for topical use which may be rinsed out or left on.

It may for example be a composition in a form chosen from the group comprising an oil-in-water or water-in-oil emulsion or a mixture of these emulsions.

According to the invention, the cosmetic or dermatological composition may for example be in a form chosen from the group comprising an aqueous or aqueous-alcoholic gel, an aqueous or aqueous-alcoholic cream and an aqueous or aqueous-alcoholic lotion. These formulations of use for the implementation of the present invention are known from the prior art by formulators. In these composition examples, it is sufficient to add agave extracts of the present invention in order to obtain a composition in accordance with the present invention.

According to the invention, the composition may be in a form chosen from a shampoo, a salve, a cream, an oil, a milk, an ointment, a powder, a solution, a gel, a serum, a balm, a butter, a lotion, a suspension, a soap or an emulsion, this form possibly being rinsed out or left on.

The extract of the present invention may be used in a cosmetic or dermatological composition alone or in combination with other cosmetically or dermatologically active or inactive substances or ingredients. The inactive substances or ingredients are those which do not act cosmetically or dermatologically. These are elements of the composition which make it possible in particular to accompany the extract, to form a particular formulation, to keep the extract active over time, this list being nonlimiting. In other words, they may be any base product which may be found in conventional cosmetic or dermatological compositions. In opposition thereto, the active substances or ingredients are those which, in the targeted cosmetic or dermatological application, have an esthetic and/or medical action.

Thus, the agave extract of the present invention may be the sole active substance or ingredient of a composition or it may be combined with other active substances or ingredients of a cosmetic or dermatological composition.

Another subject of the invention relates to a device which may be in a form chosen from a pot, a bottle, a pump bottle, a mask, a tube, a welded sachet, a spray, said device comprising an extract or a composition according to the invention.

Another subject of the invention relates to a non-therapeutic cosmetic or dermatological care process, making it possible in particular to provide an effect of improving the surface qualities of the hair or of the scalp or the growth of the hair, for example at least one effect chosen from improving the vitality, the growth, the shine, the softness of the hair, or improving the general appearance of the hair or of the scalp. The process may comprise application, to the hair and/or the scalp, of a cosmetic composition according to the invention.

Another subject of the invention relates to the use of a cosmetic or dermatological composition as defined above, for use thereof in a dermatological product intended to improve the growth and/or the appearance of the hair and/or of the scalp of a subject.

In the context of the cosmetic processes according to the invention, or of the use according to the invention, the use is understood as a non-therapeutic use, for example for treating healthy skin and hair, that is to say in particular scalps that do not exhibit a diseased state or, if the scalp does exhibit a diseased state, for a strictly esthetic use, excluding any therapeutic use. Indeed, the cosmetic and esthetic use of the invention is not associated with an inevitable therapeutic effect.

Thus, any cosmetic use and any cosmetic process according to the invention are, respectively, non-therapeutic cosmetic uses and non-therapeutic cosmetic processes that do not aim to treat a disease.

Other advantages may also become apparent to those skilled in the art on reading the examples below, given by way of illustration.

### EXAMPLES

### Example 1: Preparation of the Agave Extracts

The raw material used for the production of the fructooligosaccharides with a degree of polymerization less than or equal to 10 (commercial product Metlos®) and the inulins with a degree of polymerization greater than 10 (commercial product Metlin®) is organic blue agave harvested in the high plateaus of the state of Jalisco. After 5 to 7 years of carefully controlled growth, the agave has the maximum amount of fructans and is therefore ready to be converted.

The agave is received fresh and washed on arrival to eliminate as much foreign matter as possible. The agave is then ground in three steps to go from 40 to 100 kg of agave core to a pulpy bagasse, ready to be converted in order to extract the carbohydrates therefrom.

The crude liquor obtained from the extraction is subsequently clarified in order to eliminate any remaining color and impurities. The clarified juice is subsequently purified into two fractions: Metlos® and Metlin®. Each product is subsequently taken to a last purification step in order to eliminate all the minerals to obtain a high level of purity (>99% carbohydrates).

### Example 2: Effect of Agave Extracts Obtained in Example 1 on Epidermal Keratinocyte Renewal

The studies were carried out on normal human epidermal keratinocytes obtained from abdominoplasties, seeded in a single layer under suitable culture conditions, namely in a depleted KSFM medium (“Keratinocyte Serum Free Medium”), at a temperature of 37° C., under a moisture-saturated atmosphere containing 5% CO₂. The cells were treated with two types of agave extract (Metlin® or Metlos® at 10⁻¹%, 10⁻²% or 10⁻³%).

After 3 or 7 days of treatment (test in triplicate and reproduced twice), the cell viability was evaluated by quantification of the metabolic activity of mitochondrial dehydrogenases, by measuring the hydrolysis of MTT (“MethylThiazoleTetrazolium”).

The data in table I below relate to the percentage viability of the keratinocytes, having undergone different treatments.

The positive reference is the whole KSFM medium (with Bovine Pituitary Extract).

TABLE I
Percentage viability of keratinocytes

| Duration of | | | | | | | | |
|---|---|---|---|---|---|---|---|---|
| treatment | Base | whole | Metlin ® inulin | | | Metlos ® inulin | | |
| (days) | medium | KSFM | 10⁻¹% | 10⁻²% | 10⁻³% | 10⁻¹% | 10⁻²% | 10⁻³% |
| 3 | 100 | 159 | 133 | 131 | 141 | 136 | 132 | 132 |
| 7 | 100 | 148 | 120 | 123 | 118 | 85 | 107 | 113 |

This example shows that the addition of either one of the agave extracts into the culture medium enables the keratinocytes to proliferate up to 41% more at 10⁻³% for the Metlin® inulin and 36% more at 10⁻¹% for the Metlos® inulin. Since keratinocyte renewal is involved in the smoothing and homogeneity of the surface of the hair and of the scalp, this test validates the benefit of these extracts.

### Example 3: Effect of Agave Extracts Obtained in Example 1 on the Formation of Microtubules by Endothelial Cells

In this experiment, the cells are human umbilical vein endothelial cells (HUVEC) cultured in M200 medium (Invitrogen M200500) on matrigel (BD Biosciences 354234) for 6 hours. The cells are either in the medium (control medium) or treated by suramin (negative control) or treated by VEGF (positive control) or treated by Metlin® or Metlos® inulins (at 0.1%). After 6 hours of incubation, the pseudo-tubes formed are observed under the microscope and images are taken at random to be subsequently quantified (total surface of the network) by image analysis.

| | Control | C+ | C− | Metlin ® | Metlos ® |
|---|---|---|---|---|---|
| | medium | (VEGF) | (Suramin) | inulin | inulin |
| Concentrations | — | 50 ng/ml | 20 μM | 0.1% | 0.1% |
| Number of | 13 | 18 | 0 | 28 | 27 |
| branchings | | | | | |
| Results in % | 100 | 140 | 0 | 220 | 216 |

The treatment with Metlin® or Metlos® inulins makes it possible to stimulate the formation of pseudo-tubes by 220% at 0.1% for Metlin® and by 216% at 0.1% for Metlos®. These results therefore validate the use of the extract in a cosmetic care product aiming to stimulate hair growth.

### Example 4: Effect of Agave Extracts Obtained in Example 1 on the Migration of Endothelial Cells

The study was carried out on EPCs (circulating endothelial progenitor cells) originating from human umbilical cord blood, cultured in EGM-2 medium (Endothelial Growth medium-2).

Before the treatment, the cells are starved in 0.2% EBM-2 medium of fetal calf serum (FCS). The cells are subsequently seeded in an insert containing EGM-2 medium with 0.2% FCS, immersed in a well containing the treatment: either EGM-2 medium 0.2% FCS (negative control), or EGM-2 medium 0.2% FCS plus VEGF at 10 ng/ml (positive control), or EGM-2 medium 0.2% FCS plus the Metlin® or Metlos® inulins at 0.5% or 1%. After 5 hours of incubation, the cells inside the inserts are eliminated and those that have migrated to the other side of the insert membrane are fixed with 4% paraformaldehyde for 10 min. The cells that have migrated are subsequently counted.

| | | C+ | | | | | | | | |
|---|---|---|---|---|---|---|---|---|---|---|
| | C− | (Medium + | Metlin ® inulin | | Metlin ® inulin | | Metlos ® inulin | | Metlos ® inulin | |
| | (medium alone) | VEGF) | 0.5% | | 1% | | 0.5% | | 1% | |
| VEGF | − | + | − | + | − | + | − | + | − | + |
| (10 ng/ml) | | | | | | | | | | |
| Number of | 231 | 417 | 356 | 609 | 404 | 672 | 271 | 489 | 328 | 504 |
| cells | | | | | | | | | | |
| Results | 100% | 181% | 155% | 264% | 175% | 291% | 178% | 243% | 185% | 280% |
| relative to | | | | | | | | | | |
| C− | | | | | | | | | | |

The treatment with the Metlin® or Metlos® inulins makes it possible to stimulate the migration of human endothelial progenitor cells by 75% at 1% for Metlin® and by 85% at 1% for Metlos® in the basal condition (not stimulated by VEGF). In the stimulated condition, the treatment with the Metlin® or Metlos® inulins makes it possible to stimulate the migration of human endothelial progenitor cells by 191% at 1% for Metlin® and by 180% at 1% for Metlos®.

These results therefore validate the use of the extract in a cosmetic care product aiming to stimulate vascularization of the hair follicle and thus hair growth.

### Example 5: Evaluation of the Effect of the Inulins by Tensiometric Measurements of the Surface of the Hair

The aim of this study is to evaluate the effect of Metlin® and Metlos®, denoted respectively active agent A and active agent B, by measurements of the water contact angle on the surface of the hair.

Healthy hair is naturally protected by a surface lipid layer with hydrophobic properties.

However, the hydrophobicity of hair decreases after chemical damage, caused for example by bleaching. The aim of the present study is to measure the increase in the hydrophobicity on the surface of the hair after immersion in a solution of nourishing active agents based on fructans (inulins and/or fructooligosaccharides), due to the improvement in the hydrophobicity of the hair.

Principle of the Study

The measurement is based on the Wilhelmy method. This is a method currently used to measure the contact angle of a liquid on a single fiber. The fiber (the hair) must not be permeable to the liquid used (water) and the surface tension of this liquid must be known.

The principle consists in using high-precision scales in order to measure the force required during the immersion of the hair in the water. The contact angle is calculated from the Wilhelmy equation:

$$γ = \frac{F}{L \times cos θ}$$

with:
γ: surface tension of water: 72.8 mN/m
F: force
L: length in the wet state (measured beforehand)
θ: contact angle

As indicated above, the measurement of the contact angle requires prior determination of the wetted length of the sample. The measurement of the wetted length is carried out using a solvent with a low surface tension, hexane, which has a surface tension of 18.4 mN/m, the contact angle of which is considered to be zero.

Regarding the measurement of the contact angle, the sample is gradually immersed in hexane, and the immersion force is measured. The force F exerted just in contact with the solvent is determined by linear regression, there and by calculation, and the wetted length is determined with the Wilhelmy equation.

### Test Data

| | |
|---|---|
| Lock | Natural - treated |
| T Nat | Natural standard (European brown, supplier: Quimdis) |
| D2 | D2 − Standard |
| D2 + | D2 + 1 application by immersion, rinsed or not rinsed in |
| Treatment | the active agent solution |

Origin of the Hair: Brown European Hair.

The hair denoted “D2” or “D2 standard” is brown European hair (supplier: Quimdis) that has undergone 2 cycles of bleaching. Bleaching is the lightening of the hair's natural shade by chemical modification (alkaline oxidation processes) of the features of the melanin pigments. The highly gradual process of bleaching the pigment (solubilization by depolymerization of melanin) is preceded by an attack on the keratin and the polypeptides stabilizing the melanin pigment by the bleaching treatment (breaking of disulfide, amide, salt and hydrogen bonds). Hair that has undergone bleaching repeated twice is marked B2. We carry out this process in the laboratory by immersing the locks in a solution based on sodium persulfate and hydrogen peroxide.

The inulins or fructooligosaccharides used are Metlin® or Metlos® (NEKUTLI) and mixtures thereof, the Metlin®/Metlos® ratios of which are as follows: 100/0, 75/25, 50/50, 25/75 and 0/100.

The concentrations of the inulins and/or fructooligosaccharides in the aqueous solutions of active agents applied are 0.25%, 0.5% and 1%. This percentage corresponds to the total product in the product, that is to say either inulin, when the product does not contain fructooligosaccharides, or fructooligosaccharides when the product does not contain inulin, or the mixture of both when the product comprises inulin and fructooligosaccharides.

In some cases, the hair was rinsed after application of the inulins and/or fructooligosaccharides after 5 minutes of immersion in the aqueous solution of active agents, whereas in other cases, the hair was not rinsed.

The tensiometer used is the tensiometer Krüss K100SF.

Sampling Protocol

10 hairs per lock were measured.

For each hair, the following protocol is used:
The hair is cut from the lock using clean scissors without other handling, and handled with clean tweezers.
It is fixed on its upper portion to a sheet of paper by means of a double-sided adhesive.
For each measurement, a sample of a length of 15 mm is cut using clean scissors.
The first 3 samples are used to measure the wetted length.
The next 3 samples are used to measure the contact angle.
The sample is fixed on the adhesive surface of the sample holder over a length of approximately 5 mm (direct attachment to the sample holder without other handling). The sample must be as straight as possible, to come into contact perpendicularly with the liquid so as not to distort the measurement.

The measurement is carried out 5 mm from the bottom of the sample.

Measurement Conditions

Measurement of Wetted Length

| Parameters: | Value | |
|---|---|---|
| Rate of detection | 6 | mm/min |
| Detection sensitivity | 5.10⁻⁵ | g |
| Measurement of the speed | 3 | mm/min |
| Difference in position | 0.2 | mm |
| Depth of immersion | 5 | mm |

### Testing of the Water

The measurements are carried out with tap water which is systematically tested every day during the measurements.

### Results:

| temperature | pH | Surface tension (mN/m) |
|---|---|---|
| 23° C. | 7.4 ± 1 | 72.7 mN/m (s = 0.1) |

### Results of the Hydrophobicity Tests:

The results are given by means of a calculation of a Mann-Whitney W test to compare the medians of the two samples compared, the samples being independent. The samples compared are hair damaged by bleaching (D2) and hair damaged by bleaching (D2) and treated with the aqueous solution of active agents.

### Results of Hydrophobicity Tests 1% (Active Agent A: Inulin-Metlin®, Active Agent B: Metlos®)

| | Lock 1: | | Lock 2: | |
|---|---|---|---|---|
| | natural hair | | damaged (bleached) hair | |
| | Contact angle (°) | | Contact angle (°) | |
| References | reg | mean | reg | mean |
| mean | 102.4 | 101.5 | 78.2 | 77.8 |
| Standard deviation | 2.9 | 2.4 | 11.8 | 11.0 |
| Coeff. variation % | 2.8 | 2.3 | 15.1 | 14.1 |

| | A/B ratio | | | | | | | | | |
|---|---|---|---|---|---|---|---|---|---|---|
| | 100/0 | | 75/25 | | 50/50 | | 25/75 | | 0/100 | |
| | Non-rinsed | | | | | | | | | |
| | Lock 3 | | Lock 4 | | Lock 5 | | Lock 6 | | Lock 7 | |
| | reg | mean | reg | mean | reg | mean | reg | mean | reg | mean |
| mean | 91.8 | 90.2 | 83.7 | 83.7 | 87.1 | 85.2 | 84.5 | 82.6 | 84.4 | 82.7 |
| Standard | 7.4 | 7.8 | 13.5 | 12.3 | 13.2 | 13.6 | 11.5 | 11.5 | 15.2 | 15.7 |
| deviation | | | | | | | | | | |
| Coeff. | 8.1 | 8.7 | 16.1 | 14.7 | 15.2 | 15.9 | 13.6 | 13.9 | 18.1 | 19.0 |
| variation % | | | | | | | | | | |

| | A/B ratio | | | | | | | | | |
|---|---|---|---|---|---|---|---|---|---|---|
| | 100/0 | | 75/25 | | 50/50 | | 25/75 | | 0/100 | |
| | Rinsed | | | | | | | | | |
| | Lock 8 | | Lock 9 | | Lock 10 | | Lock 11 | | Lock 12 | |
| | reg | mean | reg | mean | reg | mean | reg | mean | reg | mean |
| mean | 90.8 | 90.5 | 87.7 | 85.7 | 87.7 | 84.7 | 83.4 | 82.1 | 87.4 | 85.9 |
| Standard | 11.6 | 11.4 | 7.0 | 7.4 | 10.6 | 11.4 | 9.5 | 8.8 | 8.3 | 8.7 |
| deviation | | | | | | | | | | |
| Coeff. | 12.7 | 12.6 | 8.0 | 8.6 | 12.1 | 13.4 | 11.4 | 10.8 | 9.5 | 10.1 |
| variation % | | | | | | | | | | |

A significant difference in relation to D2 is observed.

### Results of Hydrophobicity Tests 0.5% and 0.25% (Active Agent A: Inulin-Metlin®, Active Agent B: Metlos®)

| | Lock 1: | | Lock 2: | |
|---|---|---|---|---|
| | natural hair | | Damaged (bleached) hair | |
| | Contact angle (°) | | Contact angle (°) | |
| References | reg | mean | reg | mean |
| mean | 105.6 | 104.2 | 68.0 | 67.2 |
| Standard deviation | 3.4 | 3.5 | 15.3 | 16.6 |
| Coeff. variation % | 3.2 | 3.4 | 22.5 | 24.6 |

A significant difference in relation to D2 is observed.

| | |
|---|---|
| 50/50-0.5% | 50/50-25% |
| Contact angle (°) | Contact angle (°) |

| A/B ratio | reg | mean | reg | mean |
|---|---|---|---|---|
| mean | 77.6 | 76.8 | 69.5 | 68.0 |
| Standard deviation | 13.2 | 12.2 | 7.1 | 6.5 |
| Coeff. variation % | 17.0 | 15.8 | 10.2 | 9.6 |

P = 0.00355455**s
P = 0.0929457****ns

A significant difference in relation to D2 is observed for the 50/50 ratio at 0.5%.
Thus, a significant difference for the rinsed 50/50 ratio at 0.5% compared to the bleached control is observed, and a non-significant difference compared to the rinsed 50/50 at 1%.

### Results of Hydrophobicity Tests all Ratios Rinsed and not Rinsed at 1%+Rinsed 50/50 Tests at 0.5% and 0.25%.

A significant provision of hydrophobicity is observed for the treatments:
Rinsed at 1%: all the ratios (less for 25/75)
Rinsed at 0.5% for the 50/50 ratio
Not rinsed at 1% for all the ratios (less for 75/25 and 0/100)

### Example 6: Formulation of an Agave Extract in a Concentrated Serum for Plant-Based Hair Care

| | Percentage (% |
|---|---|
| Components (INCI name) | by weight) |
| AQUA | 54.575 |
| GELLAN GUM | 0.150 |
| SODIUM CHLORIDE | 0.075 |
| SODIUM BENZOATE | 0.500 |
| POTASSIUM SORBATE | 0.200 |
| SALICYLIC ACID | 0.150 |
| BETAINE | 2.000 |
| LACTIC ACID & AQUA | 0.150 |
| PANTHENOL | 0.200 |
| ALOE BARBADENSIS LEAF JUICE | 40.000 |
| FRUCTOOLIGOSACCHARIDES | 1.000 |
| INULIN | 1.000 |

### Example 7: Formulation of an Agave Extract in a Shampoo

| | Percentage (% |
|---|---|
| Components (INCI name) | by weight) |
| AQUA | 76.660 |
| GUAR HYDROXYPROPYLTRIMONIUM CHLORIDE | 0.150 |
| CITRIC ACID | 0.090 |
| SODIUM BENZOATE | 0.500 |
| ZINC GLUCONATE | 0.500 |
| SALICYLIC ACID | 0.050 |
| FRUCTOOLIGOSACCHARIDES | 0.125 |
| INULIN | 0.125 |
| COCO-GLUCOSIDE & GLYCERYL OLEATE | 3.000 |
| & AQUA | |
| DECYL GLUCOSIDE & AQUA | 3.000 |
| AMMONIUM LAURYL SULFATE & AQUA | 10.400 |
| COCAMIDOPROPYL BETAINE & AQUA | 4.000 |
| PARFUM | 0.400 |
| URTICA DIOICA EXTRACT & AQUA | 1.000 |
| & GLYCERIN | |

### Example 8: Formulation of an Agave Extract in a Nutritive Hair Care in the Form of Hair Conditioner

| | Percentage (% |
|---|---|
| Components (INCI name) | by weight) |
| AQUA | 87.967 |
| INULIN | 0.250 |
| FRUCTOOLIGOSACCHARIDES | 0.250 |
| PANTHENOL | 0.100 |
| SODIUM BENZOATE | 0.350 |
| BEHENTRIMONIUM CHLORIDE & ISOPROPYL | 3.000 |
| ALCOHOL | |
| STEARYL ALCOHOL | 3.500 |
| CETYL ALCOHOL | 2.000 |
| PERSEA GRATISSIMA OIL | 1.000 |
| COCOS NUCIFERA OIL | 1.000 |
| PARFUM | 0.400 |
| OLEA EUROPAEA FRUIT OIL & BETA-CAROTENE | 0.003 |
| CITRIC ACID | 0.180 |

### BIBLIOGRAPHIC REFERENCES

1. Maria de la Soledad ALONSO GUTIÉRREZ, thesis, “Valorisation de la bagasse de l'agave tequilana W. cv azul: caractérisation, étude de la digestibilité et de la fermentation des sucres” [Utilizing bagasse of Agave tequilana W. cv azul: characterization, study of digestibility and of sugar fermentation], 2005.
2. López, M. G.; Mancilla, N. A.; Mendoza-Diaz, G. (2003).: Molecular structures of fructans from Agave tequilana Weber var. azul. J. Agric. Chem. 51, 7835-7840.

1. A non-therapeutic cosmetic process for improving growth of hair and/or appearance of the hair and/or of scalp, the method comprising
applying an agave extract to the hair and/or the scalp.

2. The cosmetic process as claimed in claim 1, wherein applying said agave extract to the hair and/or the scalp gives the hair shine and/or softens the hair and/or improves the radiance of the hair.

3. The cosmetic process as claimed in claim 1, wherein said agave extract stimulates keratinocyte renewal.

4. The cosmetic process use as claimed in claim 1, wherein said agave extract stimulates the formation of microtubules and/or the migration of endothelial cells.

5. The cosmetic process as claimed in claim 1, wherein said agave extract comprises fructooligosaccharides having a degree of polymerization less than or equal to 10 and/or inulins having a degree of polymerization greater than 10.

6. The cosmetic process as claimed in claim 1, wherein said fructooligosaccharides have a degree of polymerization of 3 to 10.

7. The cosmetic process as claimed in claim 5, wherein said inulins have a degree of polymerization of 10 to 60.

8. The cosmetic process as claimed in claim 1, wherein the ratio of fructooligosaccharides to inulins is from 100/0 to 0/100.

9. The cosmetic process as claimed in claim 8, wherein the ratio of fructooligosaccharides to inulins is 50/50.

10. The cosmetic process as claimed in claim 1, wherein the agave extract is an extract of Agave tequilana.

11. A cosmetic or dermatological composition improving growth and/or appearance of hair and/or of scalp, comprising extracts of Agave tequilana and a cosmetically and/or dermatologically acceptable carrier, said composition comprising from 0.05 to 5% by weight of said extracts of Agave tequilana relative to the total weight of the composition, and said agave extract comprising fructooligosaccharides having a degree of polymerization less than or equal to 10 and/or inulins having a degree of polymerization from 10 to 60.

12. The composition as claimed in claim 11, said composition being in a form chosen from a shampoo, a salve, a cream, an oil, a milk, an ointment, a solution, a powder, a gel, a serum, a balm, a butter, a lotion, a suspension, a soap or an emulsion which may be rinsed out or left on.

13. A device in a form chosen from a pot, a bottle, a pump bottle, a mask, a spray, said device comprising a composition as claimed in claim 11.

14. A non-therapeutic cosmetic process for improving the growth and/or the appearance of the hair and/or of the scalp, comprising application to the hair and/or the scalp of a cosmetic composition as claimed in claim 11.

15. (canceled)

16. A process for preparing a cosmetic or dermatological composition as defined in claim 11, comprising mixing said agave extract and said cosmetically and/or dermatologically acceptable carrier.

17. A non-therapeutic cosmetic process for improving growth of hair and/or appearance of the hair and/or of scalp, the method comprising
applying an agave extract to the hair and/or the scalp,
wherein said agave extract stimulates keratinocyte renewal.
wherein said agave extract stimulates the formation of microtubules and/or the migration of endothelial cells.
wherein said agave extract comprises fructooligosaccharides having a degree of polymerization less than or equal to 10 and/or inulins having a degree of polymerization of 10 to 60.
wherein the agave extract is an extract of Agave tequilana.
